(19) United States
(12) Patent Application Publication
(10) Pub. No.: US 2023/0285790 A1
(43) Pub. Date: Sep. 14, 2023

(54) Portable System for the Neutralization of Chemical and Biological Agents

(71) Applicant: Soukos Robotics E.E., Larisa (GR)

(72) Inventor: Konstantinos Soukos, Larisa (GR)

(73) Assignee: Soukos Robotics E.E., Larisa (GR)

(21) Appl. No.: 18/309,983

(22) Filed: May 1, 2023

Related U.S. Application Data
(63) Continuation of application No. PCT/GR2021/000062, filed on Oct. 18, 2021.

(30) Foreign Application Priority Data
Nov. 3, 2020 (GR) 20200100663

(51) Int. Cl. A62D 3/38; A62D 3/176

(52) U.S. Cl. CPC A62D 3/38; A62D 3/176; A62D 2101/02

(57) ABSTRACT

A system and method for neutralizing chemical or biological agents present within a cavity of a piece of ordnance includes structures configured and operating for engaging the piece of ordnance between two retaining and drilling devices; boring two holes through sidewalls of the piece of ordnance to access the cavity; supply an emulsion through the one of holes to the cavity, and extract the emulsion through the second of the holes from the cavity; circulate the emulsion into a reservoir for mixing, wherein the emulsion contains chemicals to neutralize the chemical or biological agents; and continue circulating the emulsion until the chemical or biological agents have been neutralized.

### CROSS-REFERENCE TO RELATED APPLICATIONS

This patent application claims priority to International Patent Application No. PCT/GR2021/000062, which was filed on Oct. 18, 2021, and to Greek Patent Application No. 20200100663, which was filed on Nov. 3, 2020, each of which is incorporated herein in its entirety by reference.

### FIELD OF THE DISCLOSURE

The present disclosure generally relates to a portable system for the neutralization of chemical and biological agents such as those that may be contained in ammunition, ordnance, and related packages.

### BACKGROUND OF THE INVENTION

To date, there is no appropriate demilitarization method and system of different material-chemical and biological weapons that covers their widest range. The two most verified technologies that are legally acceptable are incineration and chemical inactivation, but they include issues that need to be addressed, such as the high cost of destruction and safety, as well as environmental, legal and political factors.

In particular, some studies have defined incineration as the preferred method of destruction for chemical warfare agents (CWA) due to the perceived low cost and relative simplicity of the technology. However, it is becoming clear that the incineration of chemical agents poses risks of both immediate and long-term nature, which may not be acceptable to the population. The integrity of public health and the ecosystem is threatened by the emission of materials that can escape during the incineration process, resulting in the release into the atmosphere of non-designated products of incomplete combustion. The visible incineration problems have forced US government authorities to consider alternative methods, including the chemical treatment of CWA leading to environmentally neutral products. However, this idea was rejected in the United States following research reporting that, compared to incineration, chemical neutralization processes “are slow, complex, produce excessive amounts of waste that cannot be certified as free of its precursors, and would require higher funds and operating costs.”

Problems related to the neutralization of ammunition containing chemicals combine many parameters. Firstly, because exposure to these chemicals can be harmful to humans, it is desirable that the neutralization process enhances the restriction of liquid and gaseous compounds. This can reduce the chance of liquids or vapors of these chemicals escaping from the point of neutralization where they could come into contact with the surrounding soil or groundwater, escape into the atmosphere or come into contact with humans directly or indirectly.

Secondly, when neutralizing chemical ammunition, the process preferred is the one in which the chemicals are removed from the ammunition and are substantially abated after the ammunition or ordnance is disassembled.

Thirdly, it is not desirable to move chemical ammunition from their current location to other locations where a neutralization facility is located, due to the potential damage of the ammunition and its packaging, with a high risk of chemicals escaping through the ammunition housing. This possibility is particularly strong in the long-distance transport of ammunition by land, air, or sea, in combination with the multiple handling procedures involved in such transport.

Accordingly, there is a need to transport toxic substances safely, effectively, and cost efficiently. However, complete disposal on site requires the construction of disposal units at each site. Five technologies are currently being developed and, in the future, they may provide the main methods for disposing such highly toxic chemicals. These are incineration, chemical neutralization, extremely critical water oxidation, steam gasification and plasma arc pyrolysis. All these methods involve huge costs in implementation. These costs become prohibitive, when multiplied by the number of facilities storing these chemical weapons.

### BRIEF SUMMARY OF THE INVENTION

In view of existing methods, there is a need for a portable chemical neutralization system that does not use equipment that is difficult or dangerous to transport, which is capable of enhancing the restriction of both liquid and gaseous compounds of the neutralizing chemicals, and which is also capable of effectively removing chemical agents. The prior techniques, for example, those described in U.S. Pat. Nos. 5,430,228, 5,584,071 and 5,781,868, also describe methods and systems primarily of chemical weapons hydrolysis or ozonolysis which aim to solve the same technical problems as the present invention, but these known methods have technical drawbacks. In particular, they require a combination of procedures but without the possibility of complete destruction of the precursors, in contrast to the present invention, which achieves this under the influence of closed-circuit circulation of an alcoholic solution with special chemical composition tablets, where turbulent reaction field conditions prevail.

In one aspect, the system described in the present disclosure utilizes a method, according to which neutralization of toxic substances is completed quickly and instantly without the emission of polluting liquids, solids or gaseous by-products. The system and method described herein also enables recycling the produced materials. In addition, the speed at which the toxic substances are abated or destroyed is such that it prevents a possible start of the explosive chain (high order explosion).

### BRIEF DESCRIPTION OF THE SEVERAL VIEWS OF THE DRAWING(S)

FIG. 1 is a perspective view of a system in accordance with the disclosure.

FIG. 2a is a perspective view of a device, specifically, a main operation and support unit of the system shown in FIG. 1.

FIG. 2b is a front view of the device shown in FIG. 2a, and FIG. 2c is a view of some of its internal components and configuration.

FIG. 3a is another view of the device shown in FIG. 1, and FIG. 3b is a view of some additional internal components.

FIG. 4 is a cross section view of the device of FIG. 2a.

FIGS. 5a, 5b, 6a, and 6b are assembly views of a part of the system of FIG. 1 from different perspectives.

FIG. 7 is an exploded view of the system of FIG. 1.

FIG. 8a is an assembly view of the system of FIG. 1, and FIG. 8b is an enlarged detail view of internal components of the part of the system shown in FIG. 2a.

FIGS. 9a and 9b are perspective views of drive rail components in assembled and exploded views.

FIGS. 10a and 10b are perspective views of an ammunition retaining and drilling system in accordance with the disclosure.

FIG. 11 is a cross section of a clearance area defined between the interior of the ammunition retaining and drilling system in accordance with the disclosure.

FIGS. 12a and 12b are an outline view of the system (FIG. 12a) and a partly exploded view, and a closeup view of a start/stop terminal switch (FIG. 12b) for a helical drill in accordance with the disclosure.

FIGS. 13a and 13b present in two views the operation implementation of the portable chemical and biological agents neutralization system in accordance with the disclosure.

### DETAILED DESCRIPTION OF THE INVENTION

The present disclosure relates to a portable system for the neutralization of chemical and biological agents contained in ammunition and related packages by which the neutralization-destruction of the chemical/biological agent contained in ordnance, for example, an artillery shell, takes place on site inside a closed circuit of an alcoholic solution with special chemical composition tablets, where turbulent reaction field conditions prevail. The system achieves the destruction with very high efficiency, observing all the standards of hygiene and safety as well as protection of the environment, since all kinds of gas emissions and liquid residues are avoided.

The system is portable and can be stored and transported easily and quickly by two people in backpacks to the site of the neutralization operation. This eliminates the transportation risk of dangerous and potentially unstable chemical ammunition off the battlefield. Also, after the completion of the neutralization, the system can be completely abandoned or be partly detached in order to remain hermetically sealed to hermetically contain the neutralized chemical/biological agent. The system is self-contained and, in principle, operates by engaging the artillery shell, extracting any harmful agents contained therein in a safe fashion, neutralizing the harmful agents, and thus deactivating or disarming the ordnance.

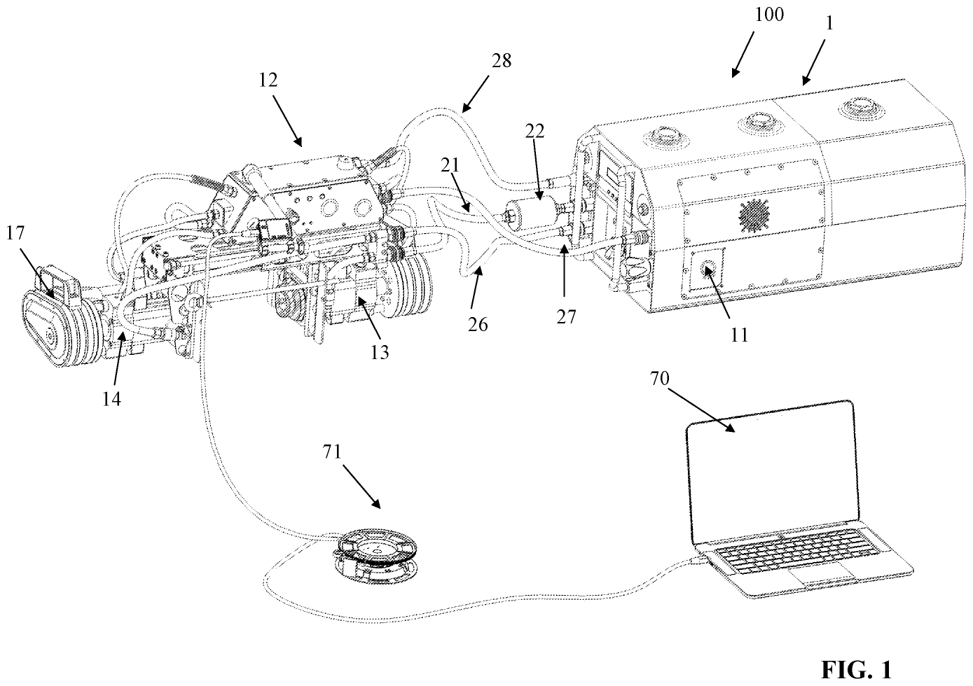

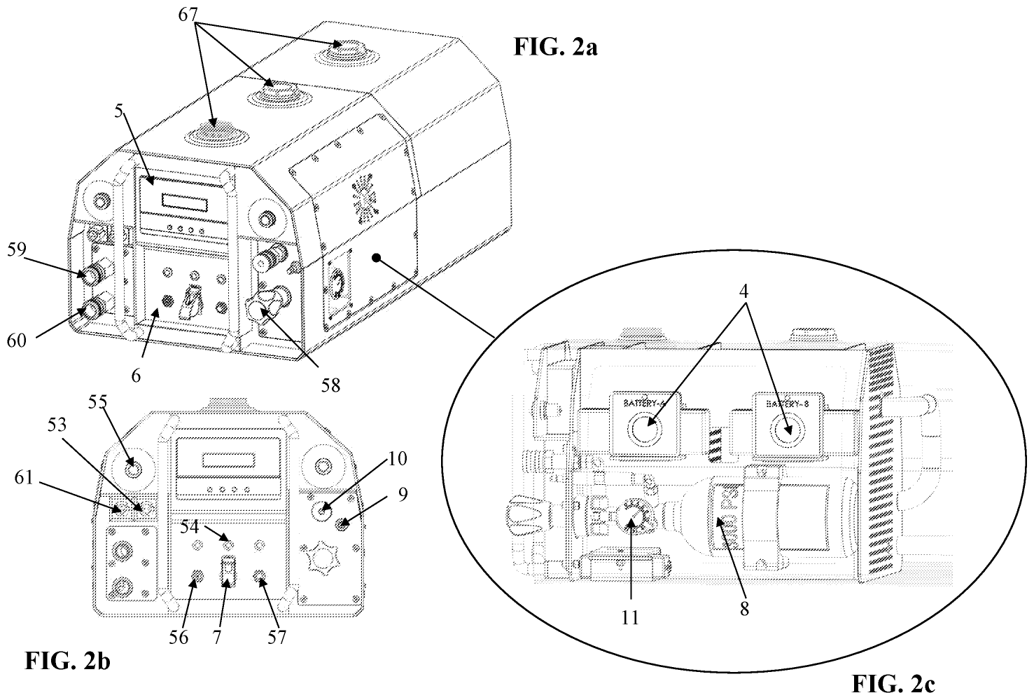

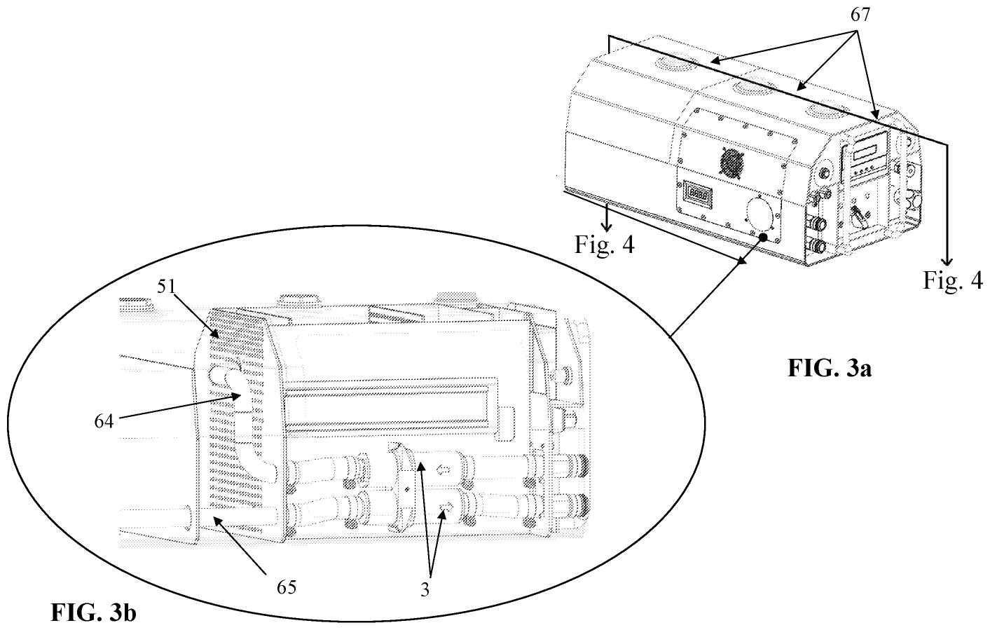

FIG. 1 presents a perspective view of a portable system (100) for the neutralization of chemical and biological agents according to the present disclosure. The system (100) includes a main operation and support unit (1), which includes and analog manometer (11, shown in FIG. 2c) that indicates a pressure of compressed air or another gas stored in the an integrated bottle (8). The system (100) also includes a rectangular configuration device (12) having two electro-pneumatic ammunition retaining and drilling systems (13), (14), each having a corresponding drive system (17). The system (100) further includes a closed monitoring circuit that optionally includes a standalone or integrated computer monitor (70) and a umbilical cable arranged in a spool (71) for remote monitoring of system operation during use. The system (100) includes a closed circuit for transferring an emulsion through connection pipes (21), (26) and a cleaning filter (22), the emulsion containing the toxic agents that are removed from the ordnance. A closed compressed air supply circuit includes a connection pipe (27), and a closed or self-contained electrical circuit are integrated with the system (100). The electrical circuit includes a reinforced spiral electric cable (28) that communicatively transfers operating parameters of the system in electronic form for control and monitoring of operation of the system (100) to and from the computer (70) during operation.

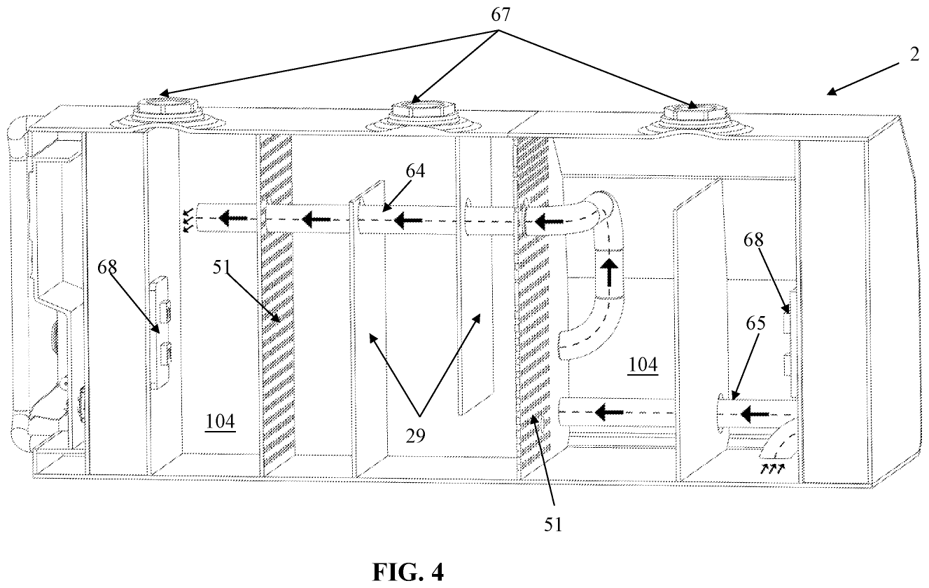

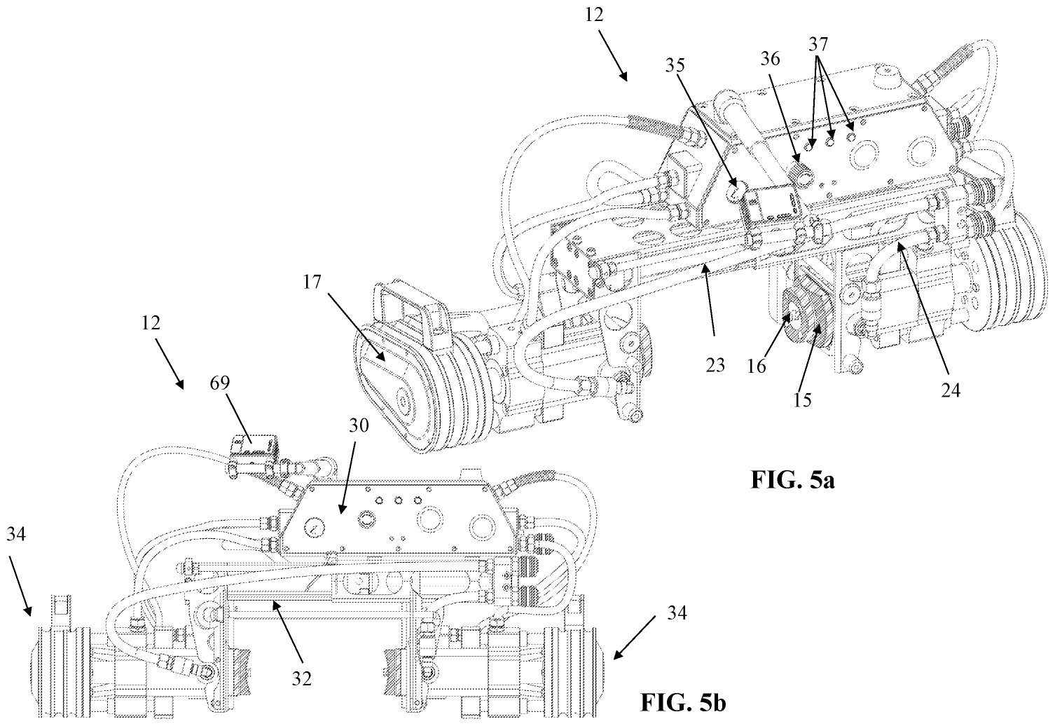

FIGS. 2a, 2b, and 2c present a perspective and side view of the main operation and support unit (1) with internal details of the included electric accumulators or batteries (4), the compressed air bottle (8) and the manometer (11). The unit (1) includes an internal reservoir (shown in FIG. 4) that contains an alcoholic solution used to neutralize toxic substances. The reservoir (104, shown in FIG. 4) is accessible through opening having lids (67) for adding or removing the alcoholic solution with controlled chemical composition tablets into and out from the reservoir (104). The unit (1) also includes a control and operation panel (6) disposed at one end of the unit (1), which includes various controls and indicators that relate to operation of the system (100) including, for example, a main switch (7), operation indicators such as a lamp of the main switch (54), operating lamps (55) of included pumps (3, shown in FIG. 3b), thermal safety indicators (56) for the pumps, and a general safety indicator lamp (57), and the like. The unit (1) also includes a compressed air outlet valve (10, shown in FIG. 2b), an outlet pressure regulating lever or knob (58), an air bottle filling valve (9), an inlet valve port (59) and an emulsion outlet port (60), a charger two-pin socket (53) and an electrical three-pin socket (61).

FIGS. 3a and 3b present another perspective view of the operation and support unit (1) with internal details of the two submersible pumps in series (3), a vertical perforated surface (51) for easy fluid inlet and outlet and a steel inlet (64) and outlet (65) pipes of the emulsion, which are the ends of the closed circuit.

FIG. 4 shows in interior detail the cylindrical cross section that includes the reservoir (104). The reservoir (104) contains the alcohol solution that neutralizes the toxic substances that are extracted from the ordnance. The reservoir (104) is arranged as a labyrinth-shaped chamber (2) and includes ultraviolet (UV) emitters (68), two vertical perforated surfaces or plates (51), and two vertical and parallel surfaces of equal thickness (29) to serve as baffles, which together create a labyrinth path for fluid circulating withing the reservoir (104). The steel inlet (64) and outlet (65) pipes circulate emulsion into and out of the reservoir (104) at both ends. Specifically, emulsion enters the reservoir (1040 from the end of the inlet pipe (64) shown on the top left portion of the reservoir (104) in the figure, and exit from the bottom right corner of the figure into outlet pipe (65). The closed circulation of the emulsion is shown with arrows.

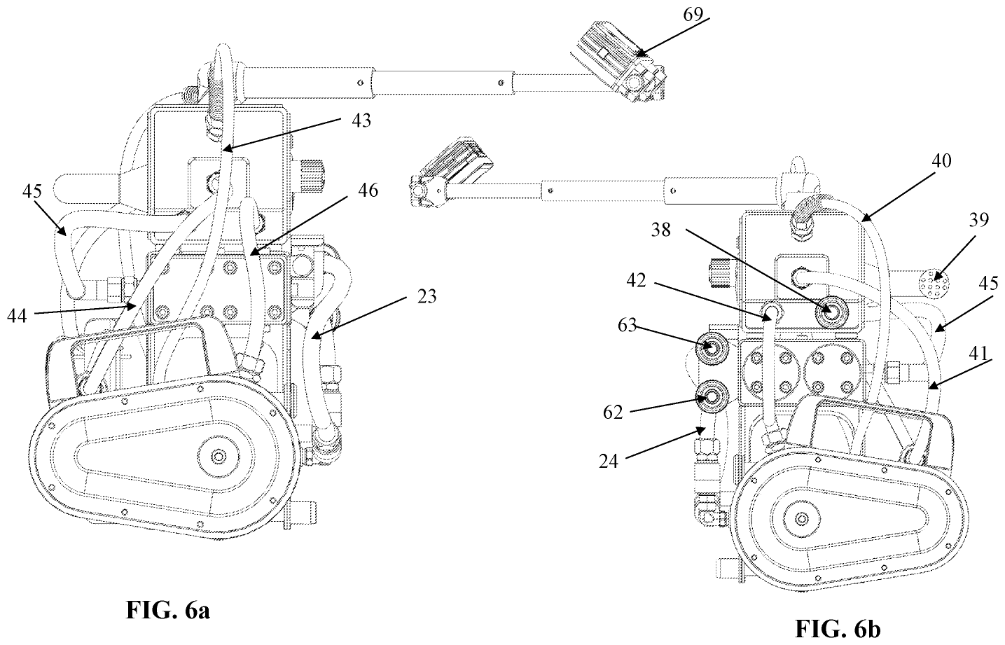

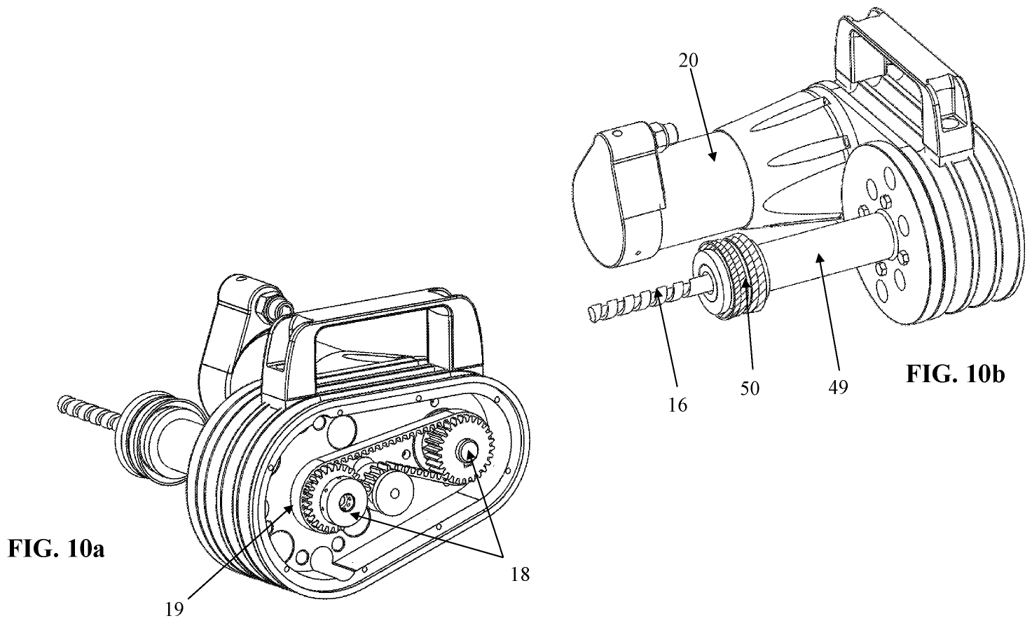

FIGS. 5a and 5b present a perspective and front view of the rectangular configuration device (12). The device (12) includes structure operating to accomplish various functions in the operation of the system (100), including engaging and securing the ordnance, drilling into the ordnance at one or two locations to extract the toxic substances, and then extracting those substances via suction and routing them to the unit (1) for neutralization. The device (12) includes a container (30), a recording camera (69) to enable remote control and operation of the system (100), for example, via the computer (70, shown in FIG. 1). The device (12) further includes a circular cross-section plastic position stabilizer (15), a helical shaped drill (16), which is also shown in FIG. 10b removed for clarity, two equally sized end drive units (34), a drive rail (32), an outlet pressure regulating lever (36), and indicator lamps (37). High pressure conduits (23) are disposed to suction and remove chemical agent contents from the ammunition and for providing the same, in the form of an emulsion with the alcohol containing solution from the reservoir (104), back to the reservoir (104) for neutralization.

FIGS. 6a and 6b present the left and right side of the rectangular configuration device (12), showing a left terminal switch connection cable (43), a left reinforced spiral electric cable (44), a high pressure drive pipe of retaining and drilling system (45) and the high pressure left drive pipe of the helical shape drill (46). Also are shown the air inlet valve (38), the emulsion inlet (62) and outlet (63) valve, the electrical power socket (39), the right terminal switch connection cable (40), the right reinforced spiral solenoid cable (41) and the high-pressure drive pipe of helical shape drill (42).

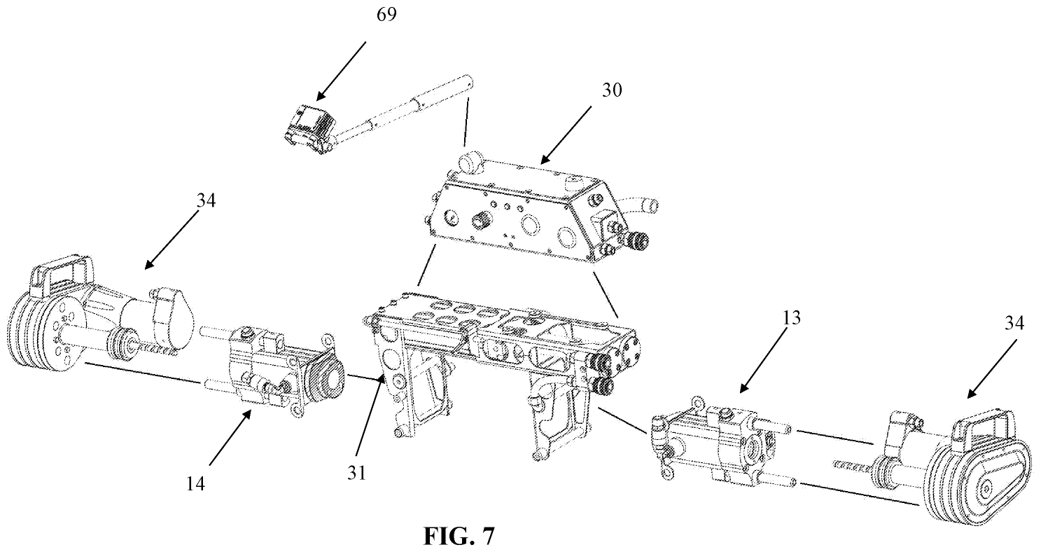

FIG. 7 illustrates the complete exploded view of the rectangular forming device (12) of the portable chemical and biological agent neutralization system with the individual construction details.

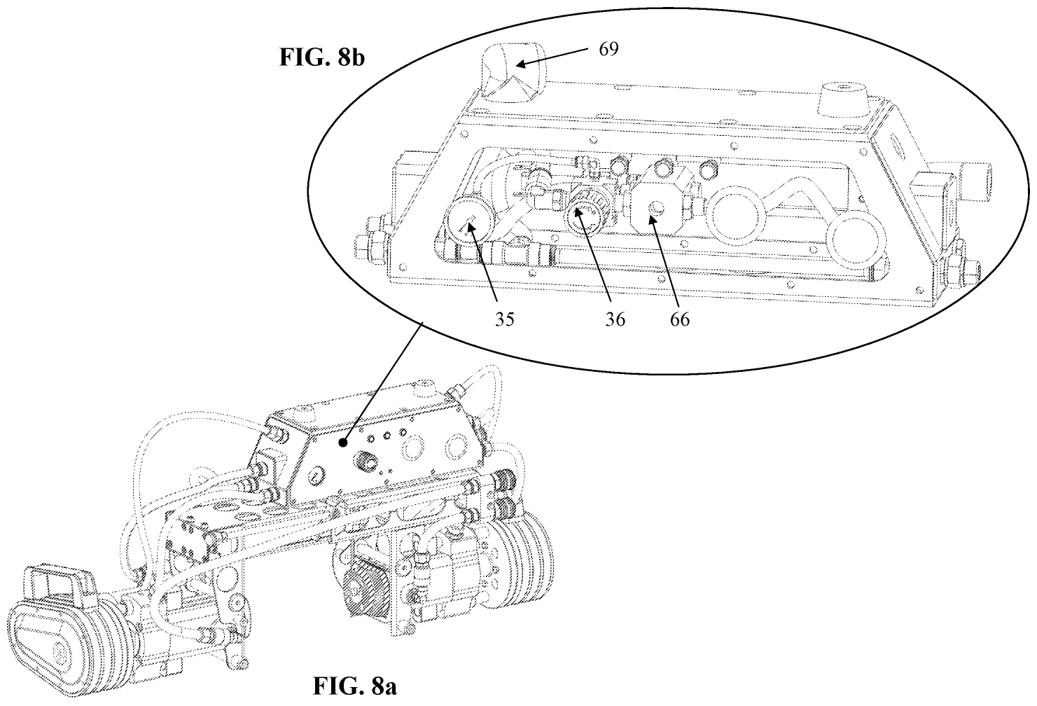

FIGS. 8a and 8b show in internal detail the trapezoidal configuration container (30) of the rectangular configuration device (12), showing the recording camera (69), the analogue manometer (35), the outlet pressure regulating lever (36) and the circuit breaker (66).

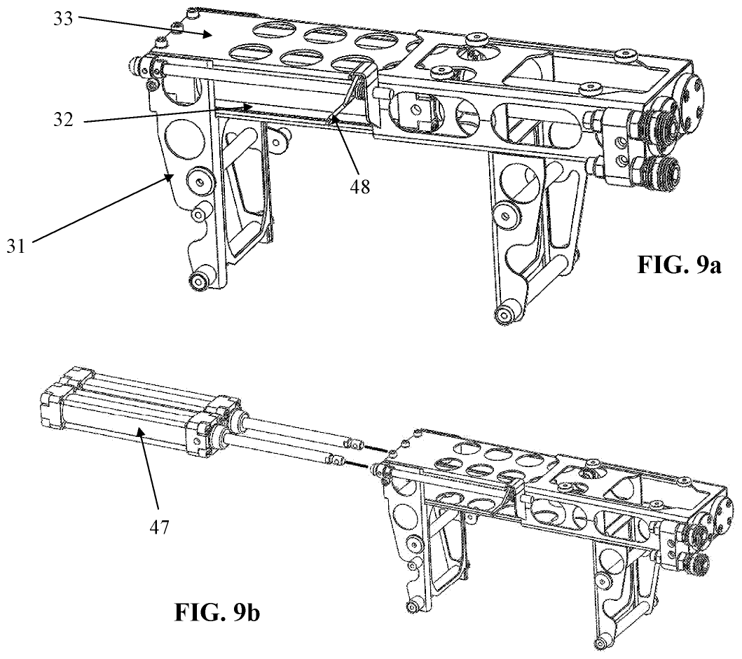

FIGS. 9a and 9b present in detail the perforated pi-shaped device (31) of the rectangular configuration device (12), with emphasis on the drive rail (32) contained on its upper horizontal side (33), the two single-acting hydraulic cylinders (47) and mechanical safety button (48). During operation, the hydraulic cylinders (47) provide a pinching force to engage the ordnance from both sides and retain the same in place while the drills bore a hold through a wall of the ordnance to enable removal of its contents for neutralization.

FIGS. 10a 10b present in detail the interior of the ammunition retaining and drilling system (13), (14) comprising a helical shape drill (16) of specific geometry and multiple point contact, a polymer made component (50), the cylindrical shaft (49) of smaller cross section, as well as the drive system (17) comprising gears (18), belts (19) and motor (20).

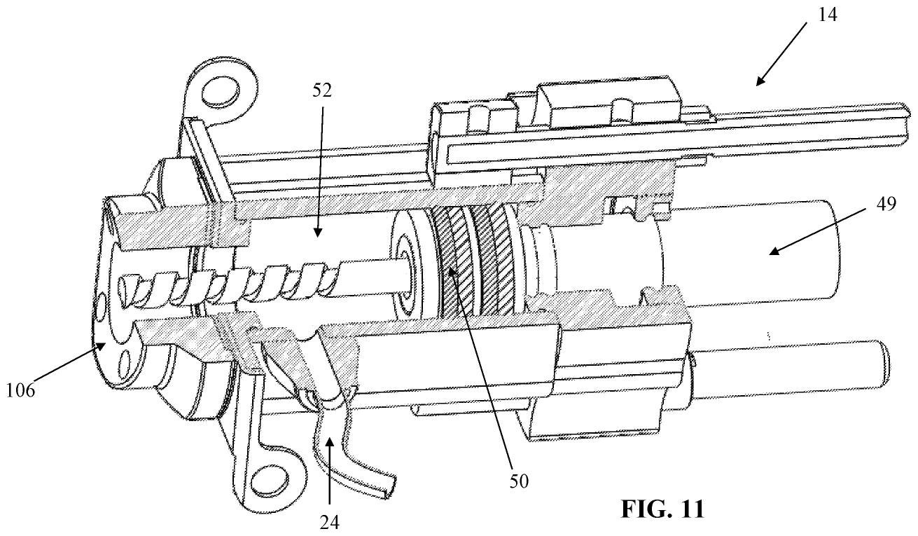

FIG. 11 presents in detail the internal illustration of the clearance (52) area defined between the interior of the ammunition retaining and drilling system (14) and the smaller cross-section cylindrical shaft (49) bearing the polymer made component (50). Also is shown is the high-pressure pipe for injection-circulation of the emulsion (24). During operation, a seal surface (106) abuts against an outer surface of the ordnance such that, when the wall of the ordnance is bored by the drill (16), toxic chemicals can only enter into clearance area (52), where they are mixed with emulsion and carried away via conduit (24) into the reservoir (104) for neutralization. Drilling using the same structure is done on two sides of the ordnance such that emulsion enters at one side, mixes with the toxic chemicals, and is carried out of the other side of the ordnance in a continuous process that removes the chemicals and also washes the interior of the ordnance with emulsion to neutralize the ordnance.

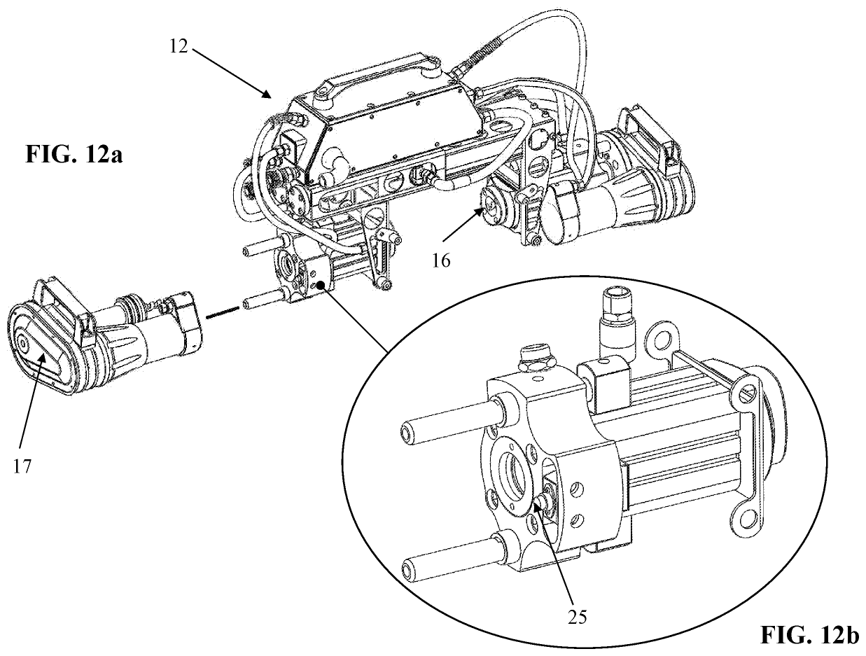

FIGS. 12a and 12b present in detail the drive system (17) showing the stop/start terminal switch (25) of the helical shape drills (16). During operation, the switch (25) will stop the drilling process once it has contacted the outer surface of the ordnance with sufficient force applied as the two sides of the drive units approach each other under action from the hydraulic actuators (47).

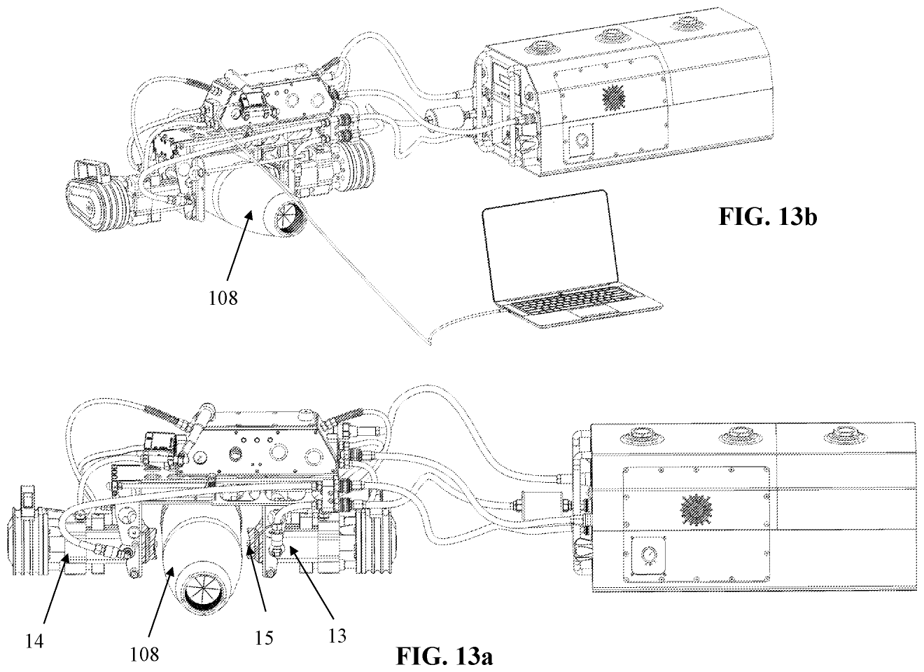

FIGS. 13a and 13b presents in two views the operation implementation of the portable chemical and biological agents neutralization system, where the ammunition (108) is placed between the circular plastic position stabilizers (15) of the retaining and drilling systems (13), (14).

A non-restrictive application of the method and the system that applies it is described below with reference to the attached figures.

The main operation and support unit (1) of the exemplary embodiment described herein is constructed as a polyhedron, of rectangular cross-section, enclosed by eight flat polygonal sides, the top side of which has a trapezoidal configuration. The choice of shape and the construction material, which is aluminum, offers a desirable combination of weight and strength, but other shapes can also be used. Aluminum also has better behavior and corrosion resistance, which makes it ideal for using the main operation and support unit (1) in an environment or on a battlefield with severe conditions. Finally, the aluminum constructions are the lightest, which favors the easy transfer of the system from the group of people to the site of the neutralization operation.

It can be manufactured in various dimensions (length, width and height). Indicatively it is mentioned that it can be made in dimensions (L×W×H)=65×35×30 cm, so that it can be easily stored in backpacks.

As shown in FIGS. 2a-2c and 4 the main unit of operation and support (1), is a solid construction that includes outside in the upper part, holes with inlet lid (67) of the alcoholic solution with the special chemical composition tablets, while inside is placed the cylindrical cross section, labyrinth-shaped chamber (2) containing the alcoholic solution with the special chemical composition tablets.

It should be noted that after the end of the first operation cycle, i.e., after the absorption of the chemical agent by the ammunition through the high pressure pipe (23) and its transfer to the cylindrical cross section, labyrinth-shaped chamber (2), then throughout the closed system that includes: the connection pipes (26), (21) of the main operation and support unit (1) with the rectangular configuration device (12) and the high pressure pipes for the absorption of the chemical agent (23) from the ammunition and for the injection-circulation of the emulsion (24), the cylindrical cross section, labyrinth-shaped chamber (2) and the clearance (52) area defined between the interior of the retaining and drilling system (13), (14) of the ammunition and the cylindrical shaft (49) of smaller cross-section, as described below, the emulsion is moving, which is the mixing product of the chemical agent and the alcoholic solution with the special chemical composition tablets.

The alcoholic solution is of specific qualitative and quantitative composition and contains a special chemical composition tablets, which are mainly calcium-based inorganic compounds, which is a white solid that can be supplied in granular form or in the form of powder and tablets. They are instantly soluble and when stored are very stable, and therefore can be supplied infrequently.

The cylindrical cross section, labyrinth-shaped chamber (2), includes two vertical perforated surfaces (51) for easy inlet and outlet of the fluid, while its interior consists of vertical and parallel surfaces (29) of equal thickness, of solid construction, which increase the flow resistance so that the emulsion does not move in parallel layers lengthwise and suddenly becomes unstable and obtains a multitude of vortices. Also, inside cylindrical cross section, labyrinth-shaped chamber (2) are placed the steel inlet (64) and outlet (65) pipes of the emulsion, which are the ends of the closed circulation circuit.

Consequently, due to the series of parallel surfaces (29) of equal thickness, which have alternating slopes, the flowing emulsion undergoes a repetitive change of direction. The conclusion that the parts contact of a fluid in turbulent flow conditions ensures their mixing, led to the development of a mixing system, which has no moving parts. In static chambers, turbulent flow conditions are ensured by the interference of obstacles in the flow of liquid, which play the same role as the reflectors in the stirred containers.

In turbulent flow there is a wide range of scales of length or sizes of vortices. Vortices that are smaller than the amount of material tend to deform it, resulting in sharp concentration gradients, which are “softened” by the molecular diffusion. In contrast, vortices that are bigger than the amount of material simply carry the material without contributing to growth. Thus, the process of mixing is analyzed in the deformation, in the stretching, in the convolution of the initial quantity. At the same time the initial volume spreads to a greater extent, resulting to the decrease of the initial concentration.

Therefore, the chemical agent that passively follows the alcoholic solution mixes rapidly along the cross section as the vortices expand and fill the entire chamber with turbulent flow.

As shown in FIG. 4, in the cylindrical cross section, labyrinth-shaped chamber (2) of the main operation and support unit (1), special UV emission devices (68) are placed, where they synergistically complete the process of chemical agents' treatment through the oxidation of the neutralized solution.

Photochemical oxidation causes the substance decomposition due to the excitation of the molecule itself by light of suitable wavelength. Depending on the part of the UV spectrum used to stimulate the molecules, photolysis is divided into 210-230 nm, 253.7 nm, 313-367 nm and 254-400 nm multicolor radiation. It is essentially the utilization of the UV-B and UV-A regions of the electromagnetic spectrum, for which special discharge lamps are used. The spectral region of ultraviolet radiation that is of interest for the applications of photolysis in the treatment of organic compounds of chemical agents is the range from 200 nm to 280 nm, i.e., part of UV-C radiation, where organic pollutants and other components (e.g., dissolved organic and inorganic compounds) absorb radiation.

The main operation and support unit (1) also includes two submersible pumps in series (3) for suctioning and injecting the emulsion containing the chemical agent with the alcoholic solution. They are high-pressure centrifugal pumps, specially designed, permanently mounted, and driven by compressed air. This hydraulic specificity gives them an excellent ability of automatic injection, even in discontinuous availability in the suction of the liquid to be transfused.

It should be noted here that the pumps act mainly as stirrers: they absorb the fluid from one side and eject it from the other. In this way, reactor conditions are carried out in the cylindrical cross section labyrinth-shaped chamber (2), resulting in faster and with maximum efficiency neutralization process.

As shown in FIGS. 2a and 2b, the main operation and support unit (1) includes internally electrical accumulators or batteries (4) to supply electricity to the system. These are rechargeable circuits that can be recharged at least 300 times before they need to be replaced. In addition to economy, they have a stable performance and maintain a constant voltage for their entire “cycle” of charging.

Under the electric accumulators is placed the compressed air supply system which includes the main compressed air storage bottle (8) and the analog manometer (11) where the amount of compressed air stored in the integrated bottle is recorded. Via the air bottle filling valve (9) takes place the compressed air refill in the bottle (8) when it is necessary.

An electronic device is also provided with an LCD screen-display (5) for charging the electric batteries, where the recharge of the electric accumulators can be done through the charger two-pin socket (53). Just below is the control and operation panel (6) which includes the main operation switch (7), as well as operation indicators, such as the main switch lamp (54), the pump operation lamps (55), the thermal safety of the pumps (56) and the general safety (57) of the main operation and support unit (1).

Also on both sides of the control and operation panel (6) are placed all the auxiliary components of the system operation such as the compressed air outlet valve (10), the outlet pressure regulating lever (58), the air bottle filling valve (9), the emulsion inlet (59) and outlet (60) valve, the charger two-pin socket (53) and the three-pin electrical socket (61) of the main operation and support unit (1).

The rectangular configuration device (12) is a special geometry manufacture in which takes place the retaining and drilling of the ammunition in order to start simultaneously the neutralization of the chemical agent through the continuous circulation of the emulsion, in a closed circuit.

It includes a fully designed electro-pneumatic system that uses electricity and compressed air as a means of operation. This involves the use of hydraulic cylinders, solenoid valves, terminal switches and indicator lights.

Specifically, the main part of the rectangular configuration device (12) is made of aluminum, while it also has parts of high hardness steel. The upper part is in the shape of a trapezoidal configuration container (30) while just below there is a perforated device in the shape of pi (31), at the lower ends of which are placed two exactly identical ammunition retaining and drilling systems (13), (14), with the only difference that the right (13) is stable while the left (14) moves on the drive rail (32) contained on the upper horizontal side (33) of the perforated pi-shaped device (31). Finally, the perforated device in the shape of pi (31) is completed with the two equally sized poles (34), of cylindrical cross-section, which are placed in series with the ends of the ammunition retaining and drilling systems (13), (14).

The trapezoidal configuration container (30) is equipped with an analogue manometer (35), an outlet pressure regulating lever (36) and operation indicator lights (37), while it also has a recording camera (69) connected to a computer monitor (70) for remote monitoring. On its right vertical side are placed the air inlet valve (38), the inlet (62) and outlet (63) valve emulsion, the electrical power socket (39) and the wiring and piping that control the right retaining and drilling system of ammunition (13), such as the terminal switch connection cable (40), the reinforced spiral electric cable (41) and the high-pressure drive pipe of helical shape drill (42). On the left vertical side is placed the wiring and piping that control the ammunition left retaining and drilling system (14), such as the left terminal switch connection cable (43), the left reinforced spiral electric cable (44), the high-pressure drive pipe of the retaining and drilling system (45) and the high pressure left drive pipe of the helical shape drill (46).

On the upper horizontal side (33) of the perforated device in the shape of pi (31) are placed the single-acting hydraulic cylinders (47) that control the electro-pneumatic retaining and drilling system (13), (14) of the ammunition. The reciprocating motion is achieved by means of the single-acting hydraulic cylinders (47), via compressed air of high-pressure drive pipe of retaining and drilling system (45). When the single-action hydraulic cylinders (47) start operating, the left retaining and drilling system (14) starts moving on the drive rail (32) and when it reaches the desired point, the ammunition holding position is secured by mechanical safety button (48), which prevents the movement from returning.

Each ammunition retaining and drilling system (13), (14) has square configuration and externally bears circular cross section plastic position stabilizers (15) for the absolute retention of the ammunition. The retaining and drilling system (13), (14) bears internally a cylindrical cross-section hole in which a cylindrical shaft (49) of smaller cross-section can be moved, in which a helical shape drill (16) of specific geometry and a multi-point contact function is installed which operates only with rotation, without impact. The helical shape drill is made of 5% cobalt alloy and high-speed steel that provide excellent surface quality, wear resistance and heat protection.

The cylindrical shaft (49) of smaller cross-section bears on its perimeter a polymer made material (50) to achieve the tightness during the movement of the cylindrical shaft (49) of smaller cross-section inside the retaining and drilling system (13), (14).

The end of each cylindrical shaft (49) of smaller cross-section that moves through the retaining and drilling system (13), (14) is joined respectively to the two equally sized poles (34) of cylindrical cross-section. At each pole (34) is mounted the drive system (17) of the helical shape drill (16) of the cylindrical shaft (49) of smaller cross section. The drive system (17) comprises gears (18), belts (19) and motor (20), which activate the propulsion function of each helical shape drill (16) towards the stable ammunition. The start of the propulsion system of the helical shape drill (16) coincides with the end of the movement of the retaining and drilling system (14), on the drive rail (32).

The function of the helical shape drill (16) of the cylindrical shaft (49) of smaller cross-section, stops when it touches the corresponding terminal switch (25) used to provide an electrical signal from the position in a clearly defined way and route.

In the clearance (52) area defined between the interior of the retaining and drilling system (13), (14) of the ammunition and the cylindrical shaft (49) of smaller cross-section bearing the polymer made component (50) which achieves the tightness or sealing contact with the outer surface of the ordnance, takes place the absorption and injection of the emulsion into and out of the cylindrical cross section, labyrinth-shaped chamber (2) of the main operation and support unit (1) through the closed emulsion circulation circuit. The start of emulsion circulation inside the closed circulation circuit coincides with the movement end of the helical shape drill (16) of the cylindrical shaft (49) of smaller cross-section.

The neutralization process according to the present invention starts with the transfer of the portable chemical and biological agent neutralization system by the group of two individuals inside backpacks to the site of the neutralization operation. Then follows the ammunition placement between the circular cross section plastic position stabilizers (15) of the holding and drilling systems (13), (14). In the meantime, the alcoholic solution with the special chemical composition tablets has been poured inside the cylindrical cross section, labyrinth-shaped chamber (2) of the main operation and support unit (1), through the holes with lid (67).

Then the necessary operating connections are made for the following circuits: a) Closed electrical circuit consisting of a reinforced spiral electrical cable (28) connecting the main operation and support unit (1) to the rectangular configuration device (12). If the batteries are charged (>24.6 V), one end of the spiral electrical cable (28) is connected to the corresponding three-pin electrical socket (61) and the other end is connected to the electrical power socket (39). B) Emulsion closed circuit comprising the connecting pipes (26), (21) of the main operation and support unit (1) with the rectangular configuration device (12). The emulsion inlet valve (59) of the main unit (1) is connected to the outlet valve (63) of the rectangular configuration device (12) and respectively the emulsion outlet valve (60) of the main unit (1) is connected to the down emulsion inlet valve (62) of the rectangular configuration device (12).

It should be noted that the connection pipe (21) having the cleaning filter (22, shown in FIG. 1) of the emulsion at one end must be connected to the inlet valve (59) of the main support and operation unit (1). The cleaning filter (22) is formed as a screen or paper filter and is mounted on the connecting pipe (21). Its housing is made of sheet steel and during the change the housing cover is unscrewed and only the filter element is replaced. The cleaning filter (22) is necessary to retain any metal particles, for example, resulting from the drilling operation of the sidewall of the ordnance, that are transported between various components of the system so as not to cause damage.

c) Closed compressed air supply circuit that includes of a connecting pipe (27), which connects the main operation and support unit (1) to the rectangular configuration device (12). The air supply connection pipe (27) is first connected to the air outlet valve (10) of the main unit (1), where the outlet pressure is adjusted to 6 bar using the outlet pressure regulating lever (58) and then to the air inlet valve (38) of the rectangular configuration device (12) where the outlet pressure is adjusted to >3 bar via the outlet pressure adjustment lever (36).

By the raise of the pressure, the operation of the single-action hydraulic cylinders (47) is activated, and the movement of the left retaining and drilling system (14) on the drive rail (32) starts. When it reaches the desired point, the ammunition holding position is secured by means of the safety mechanical button (48), which prevents any return of movement.

The main switch (7) is then activated. By activating the main switch, the lamp of the main switch (54) is also activated, indicating that the system is supplied correctly. If the outlet pressure of the rectangular configuration device (12) is above 3 bars, the circuit breaker (66) allows the start of operation of each helical shape drill (16). The start of the drive system of each helical shape drill (16) coincides with the end of the drive of the retaining and drilling system (14), on the drive rail (32).

The operation indicator lights (37) are immediately activated, signaling that through the drive systems (17), the two helical shape drills (16) begin to pierce the ammunition housing and enter its interior. The movement of the helical shape drill (16) stops when each one has penetrated to the required depth, by closing the corresponding terminal switches (25). At the same time, the polymer made component (50) of the cylindrical shaft (49) of smaller cross-section inside the retaining and drilling system (13), (14), defines the clearance (52) area inside the electro-pneumatic ammunition retaining and drilling system (13), (14) in which the emulsion moves.

Only when both terminal switches (25) are closed, the operation of both pumps (3) of the system can start automatically. The operation start of the pumps (3) is signaled by the activation of the operating lamps of the pumps (55). Then, through the connecting pipes (26), (21) and the high-pressure pipes (23), (24), a closed emulsion circulation circuit is created between the clearance (52) area and the cylindrical cross section, labyrinth-shaped chamber (2) of the main operation and support unit (1).

The above-described cycle is repeated until the electrical accumulators (4) of the system supply weaken, where is signaled the end of the neutralizing reaction of the chemical agent. Operators can then safely remove or deposit the system on site for further management—recycling.

The entire system operation is controlled by a control and operation panel (6) as well as by the computer program (70), from where the operators can monitor and control remotely each phase of the process and each subsystem. The control and operation panel (6) is equipped with monitors, phase indicators, buttons and anything else necessary to supervise and control the process.

Additionally, the applied method of the system fully complies with the term “demilitarization”, since the biological/chemical weapons are irreversibly neutralized in such a way that their reconstruction is impossible. Specifically, at the end of the process the hazardous chemical-biological substances are completely neutralized, and basic chemicals are produced: gases (such as carbon dioxide, water and nitrogen), as well as various salts (such as phosphates, chlorides, sulfates, carbonates and fluorides of calcium, sodium or potassium) depending on the specific factor.

With the application of the present invention, the complete destruction of different materials—chemical and biological weapons, takes place on site in their storage locations, since it is a portable/consumable system, with the possibility of ecological and rapid destruction, without the use of dangerous techniques, since the basic principle of the system is to limit the exposure of a minimum number of people for a minimum time, to the minimum possible amount of ammunition.

The invention focuses on an upgraded and fully ergonomic system comprising a chamber of special geometry, in the shape of labyrinth that contains the neutralization alcoholic solution with the special chemical composition tablets, the function of which is to create turbulent reactive field due to (ρ, cp, μ, D) favors the increase of the neutralization reaction rate as well as the reaction zone. The use of UV radiation also contributes to the oxidation of the neutralized solution, thus completing the process successfully and without by-products.

According to the present invention, the above-mentioned purposes, but also many more which will be better understood later, are achieved with the portable system of neutralization of chemical and biological agents from ammunition and related packages which includes two rectangular configuration units in which all the individual components and the special servo mechanisms are installed, which in combination with the use of the special alcoholic solution, bring about the complete neutralization-management of the chemical/biological agents.

The present invention relates to a portable system for the neutralization of chemical and biological agents from ammunition and related packages, comprising:

A main operation and support unit (1) bearing: A cylindrical cross section, labyrinth-shaped chamber (2) comprising the special UV emission devices (68); Two submersible pumps in series (3) for absorbing and injecting the emulsion containing the chemical agent with the alcoholic solution; Electric accumulators (4) for system power supply; Electronic device with LCD screen-display (5) for charging the electric accumulators; Control and operation panel (6) which includes the main switch (7) as well as operation indicators;

Compressed air supply system that includes: Compressed air bottle (8); Air bottle filling valve (9) and air outlet valve (10); Analogue manometer (11) which indicates the amount of compressed air stored in the integrated bottle.

A rectangular configuration device (12), bearing: Two electro-pneumatic ammunition retaining and drilling systems (13), (14), including circular cross-section plastic position stabilizers (15) and helical shape drills (16) of specific geometry and multiple point contact; Two single-acting hydraulic cylinders (47) operating the electro-pneumatic ammunition retaining and drilling systems (13) (14); Two drive systems (17) including gears (18), belts (19) and motor (20), which activate the helical shape drills (16) operation; High pressure air supply line (45) for the operation of the left ammunition retaining and drilling system (14); High pressure air supply lines (42), (46) for the operation of helical shape drills (16) of specific geometry and multiple point contact; High pressure piping for the absorption of the chemical agent (23) from the ammunition and for the injection-circulation of the emulsion (24) in the system; Terminal switches (25) with corresponding connection cables (40) (43) for stop/start of helical shape drills (16) operation.

Closed circuit of the emulsion containing the chemical/biological agent and the alcoholic solution with the special chemical composition tablets consisting of: Connection pipes (26) (21) of the main support and operation unit (1) with the rectangular configuration device (12), where the connection pipe (21) has a cleaning filter (22); Closed compressed air supply circuit consisting of a connecting pipe (27), connecting the main support and operation unit (1) with the rectangular configuration device (12); Closed electrical circuit consisting of a reinforced spiral electrical cable (28), connecting the main support and operation unit (1) with the rectangular configuration device (12); Closed monitoring and control operation circuit consisting of a recording camera (69) connected to a computer monitor (70) for remote monitoring.

System operation is controlled by a control and operation panel (6) as well as by the computer program (70), from where the operators can monitor, and control wired via a spool (71) each phase of the process and each subsystem. The control and operation panel (6) are equipped with monitors, phase indicators, buttons, and anything else necessary to supervise and control the process.

In particular, the present invention aims to present the construction and application of a portable system for the neutralization of chemical and biological agents by which the following is achieved:
a) the complete destruction of different materials—chemical and biological weapons, on site in their storage locations, since it is a portable/consumable system;
b) the neutralization is completed quickly and instantly without the emission of pollutant liquids, solids or gaseous by-products and in addition it provides the possibility of recycling the produced materials; and
c) maintaining safety for both the operators and the environment as the explosive chain is likely to start.

A portable system for the neutralization of chemical and biological agents from ammunition and related packages, according to the present invention, has many advantages.

The present disclosure provides a specific method for treating the chemical agent. In particular, the present invention successfully allows the treatment of chemical agent hydrolysates with a solution of a specific qualitative and quantitative composition that reduces the toxicity of the hydrolysate while making the component chemical precursors unsuitable for reaction during the reforming of the hydrolyzed agent.

The use of the cylindrical cross section, labyrinth-shaped chamber (2), consisting of vertical and parallel surfaces of equal thickness (29), of solid construction, increase the resistance to fluid flow so that the fluid does not move in parallel layers lengthwise and becomes suddenly unstable and display a plurality of vortices, resulting in the contact of fluid sections in turbulent flow which ensure mixing. Therefore, the emulsion mixture containing the chemical agent and the alcoholic solution is homogenized, quickly and efficiently with 100% neutralization.

The use of special UV emission devices (68) also completes the neutralization of the process since ultraviolet radiation is effective in the oxidation of organic compounds of chemical agents, the addition of chemical oxidizing reagents is not required, the risk of production, transport, storage and handling of toxic chemical reagents is reduced, contact times are very short (a few seconds) and no dangerous and toxic by-products are formed.

Also, the method, due to its uniqueness and efficiency, achieves complete treatment and recirculation of the emulsion as well as of the gaseous and solid pollutants resulting from the destruction, thus minimizing the release of gaseous pollutants into the atmosphere and the formation of solid and liquid waste.

The present invention achieves the massive and rapid destruction of chemical/biological ammunition, regardless of the initial state in which they are and the order in which they enter the system. Since the ammunition does not undergo any pre-treatment before being fed to the present invention, their destruction is instantaneous.

In addition, the present invention achieves the processing of a large class of ammunition, of all types and calibers, for many of which there is to date no suitable method of their destruction or those that exist pose a great risk to both the lives of those involved in this process and for the environment, since no measures are taken for its protection, or it is impossible to take any.

On-site neutralization activity, currently limited by the use of certain chemicals and equipment that are themselves difficult or dangerous to transport, such as high temperature incinerators and chlorine, is now possible as the system is portable and can be stored and transported easily and quickly by a group of two people in backpacks to the site of the neutralization operation.

Each subsystem and chamber of which the present invention includes may be isolated from its adjacent subsystems. The whole assembly is easily disassembled into its individual components with a parallel possibility to connect it with additional subsystems in some future use. All subsystems of the present invention are resistant to explosions and high temperatures, the elements of automation are explosion-proof, do not risk overload, while working in abnormal environmental conditions.

The use of servomechanisms, automatic electrical switches—circuit breakers and terminal switches ensures the operation of the subsystems only when necessary, resulting in energy savings and the electric accumulators remain charged for longer. Also the ability to control and operate the system remotely provides the possibility of safe and effective use.

The presence of polymer made component (50) and the circular plastic position stabilizers (15), ensure the absolute tightness of the process and the avoidance of any leaks in the surrounding area. Also, the use of mechanical safety button (48) during the ammunition stabilization process in the system prevents any accident of unintentional explosion in case ammunition falls or moves.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A portable system for the neutralization of chemical and biological agents contained in ammunition and related packages, comprising:
a main operation and support unit that includes a reservoir defining a labyrinth-shaped chamber that is adapted to contain an emulsion, at least one pump configured to circulate the emulsion through the reservoir, a power source for operating the at least one pump, a controller having a display, a control and operation panel, and a compressed air supply system that includes a compressed air bottle;
a configuration device that includes two retaining and drilling systems disposed in opposed relation, each retaining and drilling system including a position stabilizer and a helical drill, an actuator operating to move one or the two retaining and drilling system relative to the other, wherein each of the two retaining and drilling systems is adapted to sealably engage a sidewall of a piece or ordnance and to bore a hole therethrough utilizing the respective helical drill such that an interior of the ordnance is fluidly accessible and fluidly connected to an inlet and an outlet of the reservoir via conduits;
wherein a closed fluid circuit is defined for the emulsion to circulate under power from the at least one pump between the reservoir, fluid conduits supplying the emulsion to one of the two retaining and drilling systems, through an interior of the ordnance, through the second of the two retaining and drilling systems, and through additional fluid conduits to the pump and back through the reservoir.

2. The portable system of claim 1, wherein the emulsion is adapted to contain an alcoholic solution created by dissolution of chemical composition tablets.

3. The portable system of claim 1, further comprising ultraviolet (UV) emitters disposed within the reservoir.

4. The portable system of claim 1, wherein each of the two retaining and drilling systems includes a terminal switch that stops a motion of the actuator when the ordnance is retained between the two retaining and drilling systems.

5. The portable system of claim 1, further comprising a filter disposed in line with the closed fluid circuit upstream of the pump.

6. The portable system according to claim 1, wherein the main operation and support unit is shaped as a polyhedron, of rectangular cross-section, enclosed by eight flat polygonal sides, the top of which has a trapezoidal configuration, of aluminum construction.

7. The portable system according to claim 1, wherein the emulsion contains calcium-based inorganic compounds.

8. The portable system according to claim 1, wherein the reservoir includes baffles and perforated plates to promote agitation in the emulsion for improved mixing.

9. The portable system according to claim 1, wherein one of the two retaining and drilling devices is stationary and wherein the second of the two retaining and drilling devices is moveable under action of the actuator towards or away from the first of the two retaining and drilling devices such that the ordnance can be releasably engaged tightly and selectively between the two retaining and drilling devices during operation.

10. The portable system according to claim 9, further comprising a mechanical limit switch to define an engaged position of the ordnance between the two retaining and drilling devices.

11. The portable system according to claim 1, wherein each helical drill operates only by rotation, without impact.

12. The portable system according to claim 11, wherein each of the two retaining and drilling systems includes a polymer boot surrounding the helical drill, the polymer boot including a sealing surface that surrounds the helical drill and that sealably abuts against an outer surface of the ordnance.

13. The portable system of claim 12, wherein a collection chamber is defined within each polymer boot, the collection chamber being fluidly connectable to the reservoir via the conduits.

14. A method for neutralizing chemical or biological agents present within a cavity of a piece of ordnance, comprising:
engaging the piece of ordnance between two retaining and drilling devices;
boring two holes through sidewalls of the piece of ordnance to access the cavity;
supply an emulsion through the one of holes to the cavity, and extract the emulsion through the second of the holes from the cavity;
circulate the emulsion into a reservoir for mixing, wherein the emulsion contains chemicals to neutralize the chemical or biological agents; and
continue circulating the emulsion until the chemical or biological agents have been neutralized.

15. The method of claim 14, wherein circulating the emulsion is accomplished in a closed circuit under power from a pump, and wherein the closed circuit includes the cavity of the piece of ordnance.

16. The method of claim 14, wherein the emulsion contains an alcoholic solution into which chemical composition tablets are dissolved.

17. The method of claim 14, further comprising irradiating the emulsion with ultraviolet (UV) radiation within the reservoir.

18. The method of claim 14, wherein the emulsion contains calcium-based inorganic compounds.

19. The method of claim 14, wherein circulating the emulsion through the reservoir for mixing includes routing the emulsion through baffles and mixing plates.
